(19) United States
(12) Patent Application Publication
(10) Pub. No.: US 2011/0152868 A1
(43) Pub. Date: Jun. 23, 2011

(54) METHOD, DEVICE, AND SYSTEM FOR SHAVING AND SHAPING OF A JOINT

(76) Inventors: Lampros Kourtis, Berkeley, CA (US); David Myung, Santa Clara, CA (US); Michael J. Jaasma, San Francisco, CA (US)

(21) Appl. No.: 12/973,829

(22) Filed: Dec. 20, 2010

Related U.S. Application Data
(60) Provisional application No. 61/288,133, filed on Dec. 18, 2009.

(51) Int. Cl. A61B 17/16; A61B 17/56

(52) U.S. Cl. 606/80; 606/87

(57) ABSTRACT

Described herein are methods and devices useful for reaming and shaping the surfaces of a joint in a mammalian body. The reaming and shaping devices and methods are particularly useful in preparation of a joint for a minimally invasive joint replacement or resurfacing, though they may he used as part of any appropriate arthroplasty procedure.

### CROSS REFERENCE TO RELATED APPLICATIONS

This application claims the benefit under 35 U.S.C. 119 of U.S. Provisional Patent Application No. 61/288,133, filed Dec. 18, 2009, which application is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

The present invention pertains to orthopaedics and more specifically to methods and devices for joint preparation and replacement.

### BACKGROUND OF THE INVENTION

With disease or damage, the normally smooth, lubricious cartilage covering joint surfaces progressively deteriorates, exposing bone and leading to arthritic pain that is exacerbated by activity and relieved by rest. Today, patients with osteoarthritis are faced with only one of two choices: either manage their pain medically, or undergo an effective but highly bone-sacrificing surgery. Medical management includes weight loss, physical therapy, and the use of analgesics and nonsteroidal anti-inflammatories. These can be effective at reducing pain but are not curative. Other options include drugs like glucosamine or hyaluronan to replace the lost components of cartilage, but despite their extensive use in the U.S., their efficacy is still questioned.

When medical intervention fails and a patient's joint pain becomes unbearable, surgery is advised. Total joint arthroplasty is a surgical procedure in which the diseased parts of a joint are removed and replaced with new, artificial parts (collectively called the prosthesis). In this highly effective but invasive procedure, the affected articular cartilage and underlying subchondral bone are removed from the damaged joint. A variety of replacement systems have been developed, typically comprised of ultra-high molecular weight polyethylene (UHMWPE) and/or metals (e.g., titanium or cobalt chrome), or more recently, ceramics. Some are screwed into place; others are either cemented or treated in such a way that promotes bone ingrowth. These materials have been used successfully in total joint replacements, providing marked pain relief and functional improvement in patients with severe hip or knee osteoarthritis.

A large number of patients undergo total hip arthroplasty (THA) in the U.S. each year, which involves implanting an artificial cup in the acetabulum and a ball and stein on the femoral side. The goals of THA are to increase mobility, improve hip joint function, and relieve pain. Typically, a hip prosthesis lasts for at least 10-15 years before needing to be replaced. Yet despite its success as a surgical procedure, THA is still considered a treatment of last resort because it is highly bone-sacrificing, requiring excision of the entire femoral head. It is this major alteration of the femur that often makes revision replacement difficult. While this procedure has a survival rate of 90% or more in the elderly (who usually do not outlive the implant), implant lifetimes are significantly shorter in younger, more active patients. As a result, younger patients face the prospect of multiple, difficult revisions in their lifetime. Revisions are required when implants exhibit excessive wear and periprosthetic bone resorption due to wear particles, as well as aseptic loosening of the prosthesis resulting from stress shielding-induced bone resorption around the implant.

The aforementioned limitations of THA have prompted the industry to seek less bone-sacrificing options for younger patients, with the hope that a THA can be postponed by at least five years or more. One approach towards improving treatment has been to develop less invasive surgical procedures such as arthroscopic joint irrigation, debridement, abrasion, and synovectomy. However, the relative advantage of these surgical techniques in treating osteoarthritis is still controversial. An alternative to THA—hip resurfacing—has now re-emerged because of new bearing surfaces materials, such as those described in U.S. 2010/0010114 to Myung et al.

Hip resurfacing requires preparation of the joint surface to remove damaged or necrotic tissue and to shape the surface of the joint to match the shape of the device that will be implanted. Prior use of joint preparation devices and insertion of the hip resurfacing devices has required major surgery. The surgery may include removing the femur from the hip joint, elevating or removing muscles from the hip, inserting a reaming device into the bone, and removing bone and cartilage using a reamer or shaving device. These interventions may result is extensive tissue damage, pain, a long healing period, and damaged or broken bones. What are needed are tools and methods to allow a less invasive approach to orthopedic joint preparation. Such tools and methods are described in this disclosure.

### SUMMARY OF THE INVENTION

Described herein are methods and devices useful for reaming and shaping the surfaces of a joint in a mammalian body. The reaming and shaping devices and methods are particularly useful in preparation of a joint for a minimally invasive joint replacement or resurfacing, though they may be used as part of any appropriate arthroplasty procedure.

One aspect of the invention provides a method of modifying a shape of a joint surface of a mammalian joint, such as, e.g., finger joints, hip joints, knee joints, shoulder joints, or toe joints. In some embodiments, the method includes the steps of placing a cutting tool at the joint surface, the cutting tool having a cutting surface extending over more than 90° of the joint surface; moving the cutting surface with respect to the joint surface; and removing joint tissue simultaneously from more than 180° of the joint surface to thereby modify the shape of the joint surface. In some embodiments, the cutting tool has a plurality of cutting surfaces, and the moving step includes the step of moving the plurality of cutting surface with respect to the joint. Some embodiments include the additional step of assembling at least a portion of the cutting tool in situ in a join space of the joint.

In some embodiments, the joint surface is a first joint surface corresponding to a first bone, and the method further includes the step of simultaneously removing joint surface tissue from a second joint surface corresponding to a second bone with the cutting tool. Some embodiments include the additional step of forming a groove or depression in the joint surface.

In some embodiments, the cutting tool defines a central axis. In some such embodiments, moving the cutting surface includes the step of rotating the cutting surface about the central axis. In other such embodiments, the method includes the step of moving at least a portion of the at least one cutting surface toward a point on the central axis.

In some embodiments, the cutting surface has an attached end defining a pivot, and the method further includes the step of pivoting the cutting surface at the pivot. In some embodiments, the cutting tool has a plurality of cutting surfaces, and the moving step includes the step of moving the plurality of cutting surfaces.

Some embodiments include the additional steps of, before the moving steps: placing a guide pin in the joint; placing a cannulated drill on the guide pin, placing the cutting tool in the joint coaxially with the guide pin; and activating the cutting tool.

In some embodiments, the step of moving the cutting surface includes the step of gripping the joint surface with the cutting surface. In some embodiments, the step of moving the cutting surface includes the step of releasing the cutting surface from the joint surface.

Some embodiments include the additional step of distracting the joint before the placing step, and some embodiments include the additional step of compressing the joint after the placing step. Some embodiments include the additional step of applying a treatment solution, such as cooled saline, to the cutting tool after the placing step and during the activation step.

Some embodiments include the additional step of engaging a stop mechanism to prevent over-reaming of the joint surface.

Another aspect of the invention provides a method of assembling a cutting tool in a joint of a body, such as, e.g., finger joints, hip joints, knee joints, shoulder joints, or toe joints. In some embodiments the method includes the steps of placing a rotor in the joint; and, thereafter, coupling a cutting surface to the rotor, such as, e.g., by means of a slip-to-clip mechanism. The method may also include the step of creating a path through a bone in the body before the placing step, the path configured to accept the rotor. Some embodiments include the additional step of placing a centering pin though a bone in a body, the centering pin configured to align the rotor with the centering pin.

Some embodiments include the additional step of distracting the joint before the coupling step. In embodiments in which the joint has first and second bones, the distracting step may include the steps of placing a distractor linear actuator module in the joint, the distractor linear actuator module comprising first and second bone attachment portions; attaching the first bone attachment portion to the first bone; attaching the second bone attachment portion to the second bone; and applying a force between the two bone attachment portions to cause the joint to distract. In some embodiments, the step of attaching the first and second attachment portions includes the step of inserting first and second bone screws.

Some embodiments include the additional step of attaching a distractor linear actuator module to the bone screw. Such embodiments may also include the step of attaching a drill assembly to the distractor.

Yet another aspect of the invention provides a device configured to modify a shape of a joint surface of a mammalian joint, such as, e.g., finger joints, hip joints, knee joints, shoulder joints, and toe joints. In some embodiments, the device includes a cutting tool having a cutting surface adapted to extend longitudinally over more than 90° of the joint surface and to move with respect to the joint surface to remove joint tissue simultaneously from more than 180° of the joint surface as it moves.

In some embodiments, the joint surface is a first joint surface corresponding to a first bone, and the cutting tool is configured to simultaneously remove joint surface tissue from the first joint surface and from a second joint surface corresponding to a second bone.

In some embodiments, the cutting surface has a first facial surface configured to face a first joint surface and to reshape the first joint surface and a second facial surface configured to face a second joint surface and to reshape the second joint surface, and the first and second facial surfaces have a matching geometric shape, such as, e.g., a sphere, tapered cylinder, chamfered cylinder or ellipse. In other embodiments in which the cutting surface has a first facial surface configured to face a first joint surface and to reshape the first joint surface and a second facial surface configured to face a second joint surface and to reshape the second joint surface, the second facial surface has a different geometric shape than the first facial surface.

In some embodiments, the cutting surface has a protrusion configured to further modify the shape of the joint surface to create a depression. In some embodiments, the cutting surface has an ellipsoid arc (such as, e.g., a spherical arc) extending longitudinally from between 91° and 125° from an end of the cutting surface.

In some embodiments, the device is further configured to provide a distraction force to the joint. In some embodiments, the device is further configured to deliver power to move the cutting surface. In some embodiments, an orientation of the cutting surface defines a central axis and the cutting surface is configured to rotate around the central axis.

In some embodiments, the cutting surface is further configured to grip the joint surface such as, e.g., by continuously gripping the joint surface during a period of device use. The device may also include a control system configured to apply a force to the cutting surfaces to cause the cutting surfaces to grip the joint surface, such as a hydraulic, pneumatic, and mechanical control system. The control system may also be configured to change an amount of the force while the device is in use. In some embodiments, the mechanical control system may include a spring.

In some embodiments, the cutting surface has a coupling end, a free end and a collar region on the free end.

In some embodiments, the device includes a wire-rope and a wire-rope tensioner, the tensioner configured to control a tension of the wire-rope and the wire-rope configured to control a position of the cutting surface. The wire-rope tensioner may he manually controlled in some embodiments.

In some embodiments, the cutting surface has an attached end defining a pivot and the cutting surface is configured to pivot at the pivot. In some embodiments, the cutting tool defines a central axis, a portion of the cutting surface configured to move toward a point on the central axis.

Some embodiments of the device are configured to deliver a treatment solution (such as, e,g., cooled saline) into the joint.

Some embodiments of the invention are further configured to deliver a compression force into the joint, such as by using hydraulic, pneumatic, and mechanical control.

Some embodiments of the invention include a guide-pin configured to center the cutting surface on the guidepin.

In some embodiments of the invention, the cutting surface includes an abrasive and/or teeth. The cutting surface may be configured to extend laterally over 1-100% of the joint surface.

Some embodiments of the invention also include a stop mechanism configured to prevent over-reaming of the joint surface.

Another aspect of the invention provides a cutting tool system for modifying a shape of a joint surface of a mammalian joint and configured for assembly in the joint. In some embodiments, the system includes a rotor and a cutting surface configured to removably couple with the rotor, to extend longitudinally over more than 90° of the joint surface, and to move with respect to the joint surface to thereby remove joint tissue simultaneously from more than 180° of the joint surface. Some embodiments of the invention also include a distractor linear actuator module having a supply of power and configured to cause the rotor to rotate.

In some embodiments, the joint surface is a first joint surface corresponding to a first bone, and the cutting surface has a first facial surface configured to face the first joint surface and configured to reshape the first joint surface and a second facial surface configured to face a second joint surface and configured to reshape the second joint surface, the first and second facial surfaces configured to move with respect to the first and second joint surfaces to thereby remove joint tissue simultaneously from the first and second joint surfaces. In some such embodiments, the first and second facial surfaces have a matching geometric shape, such as, e.g., a sphere, tapered cylinder, chamfered cylinder or ellipse. In other such embodiments, the second facial surface has a different geometric shape than the first facial surface. In some embodiments, at least one of the first or second facial surfaces comprises a protrusion configured to further modify the shape of the first or second joint surface to thereby create a depression.

In some embodiments, the cutting surface includes an ellipsoid arc (such as a spherical arc) extending longitudinally from between 91 and 125° from an end of the cutting surface.

In some embodiments, the cutting tool is configured to provide a distraction force to the joint for use to distract the joint. In some such embodiments, control of the distraction force may be hydraulic, pneumatic, and/or mechanical.

Some embodiments are further configured to deliver power to cause the cutting surface to move, such as, e.g., by means of rotation of the rotor.

In some embodiments, an orientation of the cutting surface defines a central axis and the cutting surface is configured to rotate around the central axis. Some embodiments of the invention have a plurality of cutting surfaces.

In some embodiments, the cutting surface is further configured to grip the joint surface. The cutting surface may be configured to continuously grip the joint surface when the system is in use, such as by using a control system configured to apply a force to the cutting surface to cause the cutting surface to grip the joint surface. The control system may be hydraulic, pneumatic, and/or mechanical (e.g., cam-trigger and/or spring).

In some embodiments, the cutting tool system may be configured to deliver a treatment solution, such as, e.g., cooled saline, to the joint surface.

In some embodiments, the cutting tool system is further configured to deliver a compression force into the joint, such as by a hydraulic, pneumatic, and mechanical compression force.

Some embodiments of the invention include a guide-pin configured to center the cutting surface on the guidepin.

In some embodiments of the invention, the cutting surface includes an abrasive and/or teeth. The cutting surface may be configured to extend laterally over 1-100% of the joint surface.

Some embodiments of the invention also include a stop mechanism configured to prevent over-reaming of the joint surface, such as, e.g., a limiting nut on the blade rotor; a mating element on the rotor configured to mate with a portion of the cutting surface to cause the rotor to stop moving. Some embodiments have two cutting surfaces, and the stop mechanism comprises mating aspects of the cutting surfaces.

Still another aspect of the invention provides a method of modifying a shape of a joint surface of a mammalian joint, such as finger joints, hip joints, knee joints, shoulder joints, and toe joints. In some embodiments, the method includes the steps of placing a cutting tool at the joint surface, the cutting tool having a central axis and a cutting surface extending over the joint surface, the cutting surface forming a blade angle with the central axis; and simultaneously rotating the cutting surface about the central axis and changing the blade angle to remove joint tissue and thereby modify the shape of the joint surface.

In embodiments in which the cutting surface is a first cutting surface, the cutting tool may further include a plurality of cutting surfaces each forming a blade angle with the central axis, in which embodiment the moving step includes the step of simultaneously rotating the plurality of cutting surfaces about the central axis and changing their blade angles to remove joint tissue and thereby modify the shape of the joint surface.

Some embodiments include the step of assembling at least a portion of the cutting tool in situ in a joint space of the joint.

In some embodiments, the joint surface is a first joint surface corresponding to a first bone, and the method further includes the step of simultaneously removing joint surface tissue from a second joint surface corresponding to a second bone with the cutting tool.

Some embodiments add the step of forming a groove or depression in the joint surface.

In some embodiments, the cutting surface extends over more than 15° of the joint surface, and the rotating step further includes the step of simultaneously removing joint tissue from more than 30° of the joint surface to thereby modify the shape of the joint surface.

In some embodiments, the cutting surface extends over more than 90° of the joint surface, and the rotating step further includes the step of simultaneously removing joint tissue from more than 180° of the joint surface to thereby modify the shape of the joint surface.

In some embodiments, the cutting surface has an attached end defining a pivot, and the moving step includes the step of pivoting the cutting surface at the pivot.

In some embodiments, the cutting tool defines a central axis, and the rotating and changing step further comprises moving at least a portion of the at least one cutting surface toward point on the central axis.

Some embodiments provide the additional steps of, before the rotating step: placing a guide pin in the joint; placing a cannulated drill on the guide pin; placing the cutting tool in the joint; and activating the cutting tool.

In some embodiments, the simultaneously rotating and changing step further includes the step of causing the cutting surface to grip the joint surface. In some such embodiments, the causing is controlled automatically, and in some embodiments the causing is controlled manually.

Some embodiments provide the additional step of distracting the joint before the rotating and changing step. Some embodiments provide the additional step of compressing the joint before the rotating and changing step.

Some embodiments provide the additional step of applying a treatment solution, such as, e.g., cooled saline, to the cutting tool. Some embodiments include the step of engaging a stop mechanism configured to prevent over-reaming of the joint surface after the rotating and changing step.

Yet another aspect of the invention provides a cutting tool system for modifying a shape of a joint surface of a mammalian joint. In some embodiments, the system includes a cutting surface; a rotor operatively connected to the cutting surface and adapted to rotate the cutting surface about a central axis; and a blade angle actuator operatively connected to the cutting surface and adapted to change an angle of the cutting surface with respect to the central axis as the surface is rotated by the rotor.

Some embodiments also have a distractor shell module having a supply of power and configured to rotate the rotor. In some embodiments, the cutting surface is configured to removably couple with the rotor.

In some embodiments, the cutting surface is configured to extend longitudinally over more than 15° of the joint surface. In some embodiments, the cutting surface is configured to laterally cover from 1-100% of the joint surface.

In some embodiments, the cutting surface is configured to extend longitudinally over more than 90° of the joint surface. In some embodiments, the cutting surface is adapted to remove joint tissue is removed simultaneously from more than 180° of the joint surface.

In some embodiments, the joint surface is a first joint surface and the cutting surface has a first facial surface configured to face a first joint surface and to reshape the first joint surface and a second facial surface configured to face a second joint surface and to move simultaneously with respect to the first and second joint surfaces to thereby simultaneously remove joint surface tissue from the first and second joint surfaces. In some such embodiments, the first and second facial surfaces have a matching geometric shape, such as, e.g., a sphere, tapered cylinder, chamfered cylinder or ellipse. In other such embodiments, the second facial surface has a different geometric shape than the first facial surface. In some embodiments, at least one of the first or second facial surfaces comprises a protrusion configured to further modify the shape of the first or second joint surface to thereby create a depression.

In some embodiments, the cutting surface has an ellipsoid arc (such as spherical arc) extending longitudinally between 15° and 125° from an end of the cutting surface. In some such embodiments, the arc may end from between 91° and 125° from the end of the cutting surface.

Some embodiments are further configured to provide a distraction force to the joint for use to distract the joint. Such embodiments may also provide a control of the distraction force selected from the group consisting of hydraulic, pneumatic, and mechanical control. Some embodiments are further configured to deliver power to cause the cutting surface to move. The cutting tool system may have a plurality of cutting surfaces.

In some embodiments, the cutting surface is configured to grip the joint surface. The cutting surface may be configured to continuously grip the joint surface when the system is in use. The system may have a control system configured to apply a force to the cutting surface to cause the cutting surface to grip the joint surface. The control system is selected from the group consisting of hydraulic, pneumatic, and mechanical (e.g., cam-trigger and/or spring control).

In some embodiments, the system is further configured to deliver a treatment solution (such as cooled saline) to the joint surface.

Some embodiments are further configured to deliver a compression force into the joint. In such embodiments, control of the compression force may be hydraulic, pneumatic, or mechanical control.

Some embodiments of the invention also have a guide-pin configured to guide the cutting surface.

In some embodiments of the invention, the cutting surface has an abrasive and/or sharp protrusions.

Some embodiments have a stop mechanism configured to prevent over-reaming of the joint surface. In some embodiments, the cutting surface has an attached end defining a pivot and the cutting surface is configured to pivot at the pivot. In some embodiments, the cutting tool defines a central axis, at least a portion of the cutting surface being configured to move inward relative to a point on the central axis.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which.

In the drawings:

FIG. 1 shows a composite view of a reaming and shaper device in a hip joint according to one aspect of the disclosure. The figure shows a partial outside view superimposed over a longitudinal cross-sectional view.

FIG. 2 shows the reaming device of FIG. 1 with the joint space of the hip distracted.

FIG. 3 shows a detail view of the head of the reaming device of FIGS. 1-2 with one of the cutting blades (cutting surfaces) being inserted into the device head and two other cutting blades (cutting surfaces) already in place.

FIGS. 4-11 show steps in the assembly in the hip of the reaming device of FIG. 1.

FIG. 4 shows a path drilled through the femur and acetabulum and a centering pin being inserted in the acetabulum.

FIG. 5 depicts insertion of a blade rotor over a centering pin in the femur.

FIG. 6 depicts insertion of a trochanter screw over the blade rotor in the femur.

FIG. 7 shows attachment of the distractor linear actuator module to the pelvic centering pin and the trochanter screw.

FIG. 8 shows a cross-section view through the distractor linear actuator module and pressurization of the distractor chamber to distract the hip joint space.

FIG. 9 shows arthroscopic porting of the first cutting blade in the synovial capsule.

FIG. 10 shows coupling of a cutting blade onto the blade rotor.

FIG. 11 shows the reamer with three cutting blades coupled to the blade rotor.

FIG. 12 shows pressurization of the compression chamber in the distractor linear actuator module and compression of the hip joint space.

FIG. 13 depicts the drill attached to the blade rotor, delivery of torque to the petals, and simultaneous reaming of femoral head and acetabular surface.

FIG. 14 shows the joint after reaming is complete. The last cutting blade is being arthroscopically removed.

FIG. 15 shows a step in the disassembly of the reamer after the distractor linear actuator module has been removed.

FIG. 16 shows a femoral head shaper with arc shaped cutting blades with an abrasive surface on a femoral head and a spring control mechanism according to one aspect of the invention.

FIG. 17 shows another femoral head shaper on a femoral head at the end of a reaming procedure with cutting blades with small cutting teeth in a stopped position according to one aspect of the invention.

FIGS. 18A and B show preparation of a joint surface for treatment with a device described herein.

FIG. 19 shows a femoral head shaver with three cutting blades and a pressure activated control mechanism.

FIG. 20 shows a femoral head shaver with three cutting blades, a hydraulic control mechanism, and a hydraulic control lever.

FIG. 21 shows a femoral head shaper being placed on a femoral head with the cutting blades in an open configuration.

FIG. 22 shows the femoral head shaper of FIG. 21 with the cutting blades closed over the femoral head.

FIG. 23 shows the femoral head shaper of FIG. 22 moved to a different position on the femoral head according to one embodiment of the invention,

FIG. 24A-B show an inside view of the headpiece and cutting surface of a femoral head shaper like that shown in FIG. 22.

FIG. 25A-C show an outside view of the headpiece, cutting surface and attachment pin of a femoral head shaper like that shown in FIG. 22.

FIG. 26 shows a detail view of a femoral shaper similar to the device in FIG. 19 with a spring mechanism, pressure sheath and rotor feature to control the cutting surfaces.

FIG. 27 shows an inside/bottom view of the femoral shaper shown in FIG. 21 with cutting surfaces having sharp cutting surfaces and a protrusion.

### DETAILED DESCRIPTION THE INVENTION

The present invention includes a method, device, and system to quickly prepare a joint surface for subsequent surgery. Preparation may be done in one step and may not require open exposure of the joint. The benefits include an option for a minimally invasive (arthroscopic) surgery for joint preparation and coordinated reaming and smoothing of the joint surfaces so that the two sides of the joint match. For brevity, the invention is shown configured for use in a hip joint, but may be used on any suitable movable joint (e.g., ball-and-socket, condyle or hinge-structure joints, such as finger, hip, knee, shoulder, toe, etc.).

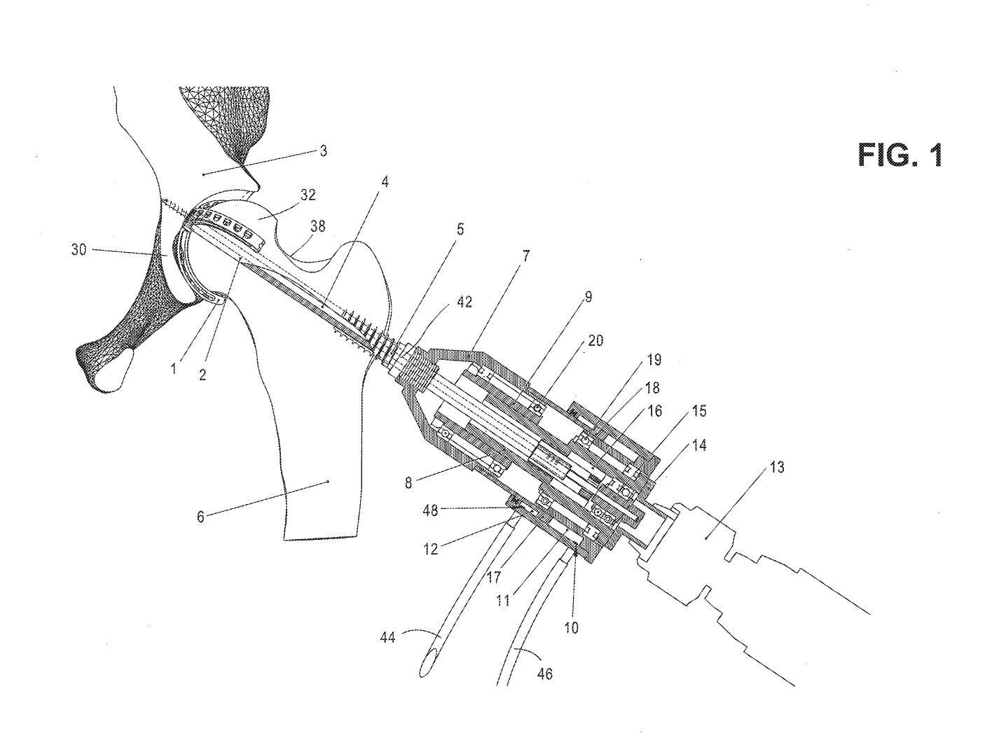

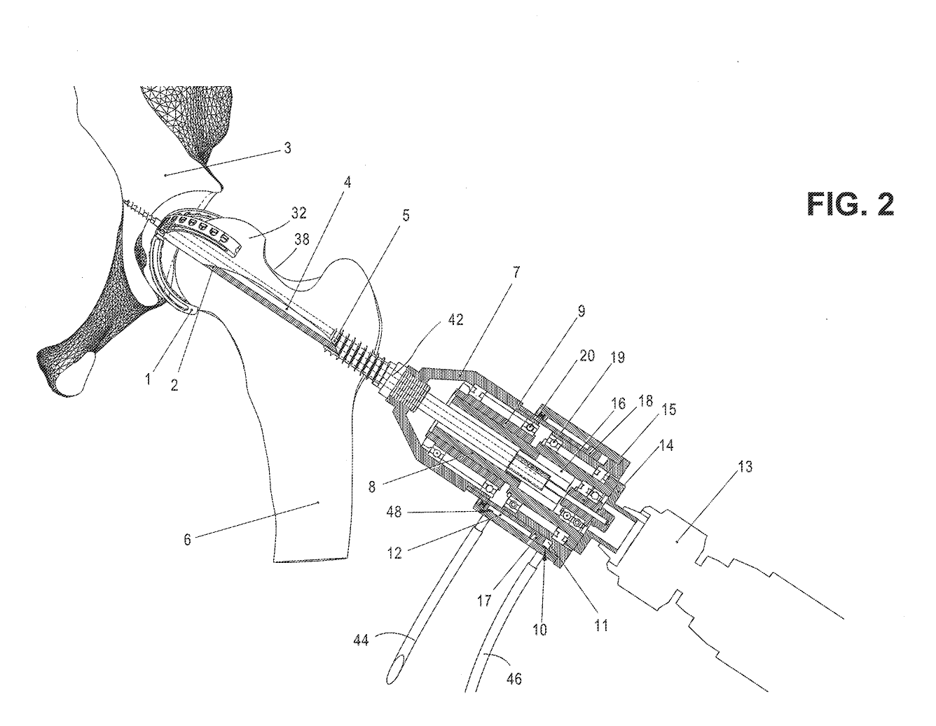

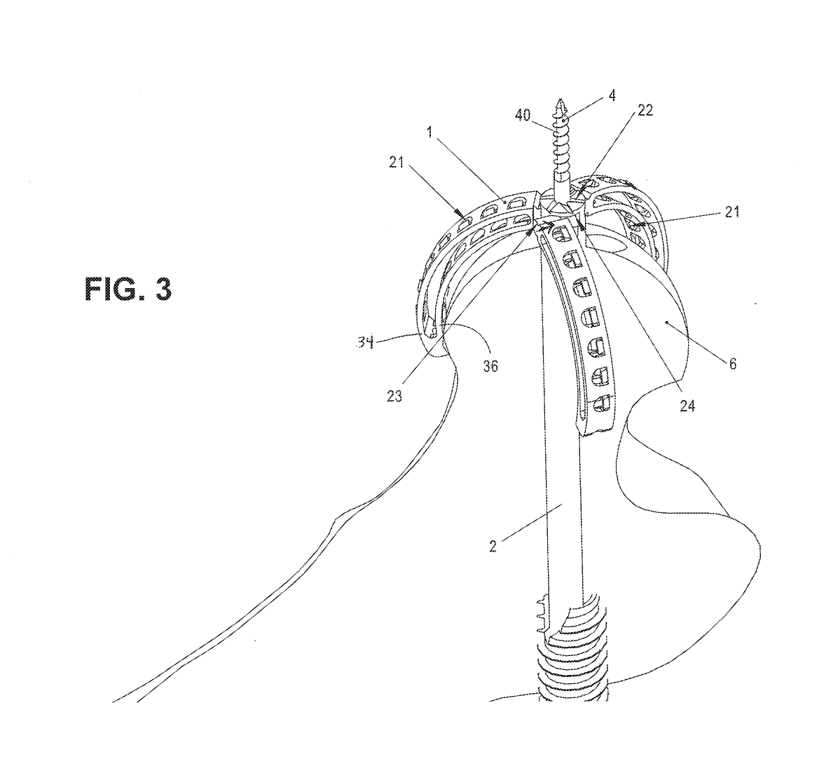

A combined reaming and shaping device configured for a hip joint according to one aspect of the disclosure is shown in FIGS. 1-3. A shaping device according to the disclosure may function to shape, etch, indent, notch, rasp, ream, score, shave, or otherwise modify a shape of a joint surface. FIG. 1 shows a composite view of a reaming and shaper device in a hip joint according to one aspect of the invention. The figure shows a partial outside view superimposed over a partial longitudinal cross-sectional view. FIG. 2 shows the reaming device of FIG. 1 with the joint space of the hip distracted. FIG. 3 shows a detail view of the head of the reaming device of FIGS. 1-2 with one of the cutting surfaces being inserted into the device head.

The reaming and shaping end of the device is able to rotate about a central device axis. In one embodiment, the device may be configured to ream an acetabular side of a joint at the same time it shapes a femoral head. The device may be trapped between two joint surfaces to cause the device to simultaneously shave both sides of the joint. A distractor may provide a compressive force to bring the femur close to the acetabulum to generate a reaming reaction force that traps the cutting surfaces between the two joint surfaces as the reaming and shaping end of the device rotates. In one embodiment, the reaming and shaping end of the device may be able to oscillate (e.g. change rotational direction about a central axis). The device may oscillate after performing less than a full revolution, or may oscillate after performing one or more revolutions.

Cutting surfaces (e.g., blades or petals) 1 are shown in the joint space of a joint attached to the blade rotor 2, the distractor linear actuator module 7, and the drill 13. The cutting surface may be shaped as a long or short, narrow or wide blade. The cutting surface may be arc shaped and may extend longitudinally along the surface of the femoral head. In one embodiment, they may extend 91° to 125°. The device may have one or a plurality of cutting surfaces. The cutting surfaces are able to rotate about a central axis (e.g., the axis of the bone, axis of the blade rotor, or an axis defined by the cutting surface and to remove thin slivers of bone, cartilage or other material from the joint surface as they rotate. The cutting surface may have surface roughness or sharpness in any form that may cause material to be removed from the joint surface. The cutting surface may have, e.g., sharp, pointed teeth with or without directionality or may have an attached or embedded abrasive material.

The cutting surfaces may have both convex and concave facial surfaces. FIG. 3 shows one facial surface 34 configured to face the end of the pelvis and the other facial surface 36 facing the femur 6. The cutting surfaces 1 may have one or a plurality of cutting edges or portions 21 on one or on both facial surfaces 34, 36. Having cutting edges or portions on both facial surfaces 34, 36 may allow the two sides of the joint to be reamed and shaped simultaneously. It may also allow the two sides of the joint to be reamed or shaped sequentially using a single deployed device.

The cutting surfaces may remove joint surface tissue from more than 180° of the joint surface to modify the joint tissue. The profile (shape) of the cutting surfaces 1 matches the desired joint surface profile. The concave side may have a same or different profile (e.g., spherical, tapered cylinder, chamfered cylinder) than the convex side (e.g., spherical, elliptical). The cutting surface 1 may carry one or more features that may create a groove or depression on the joint surface upon rotation of the cutting surface.

The cutting surface(s) 1 can be attached to the blade rotor 2 by any suitable means that allows them to pivot with respect to the blade motor and move with respect to the joint to shape and shave joint tissue. The attachment mechanism may allow the cutting surfaces 1 to be easily attached or removed, e.g., for assembly or disassembly of the cutting surfaces from the blade rotor 2. In one embodiment, the cutting surface 1 is attached to the blade rotor 2 using a slide-to-clip mechanism by means of a key 23 on the cutting surface coupling with a keyway 24 on the blade rotor 2. A plurality of cutting surfaces on a reaming and shaping device may be symmetrically disposed around a central (blade, device, or cutting surface) axis.

One end of the blade rotor 2 may be removably coupled with the cutting surface(s). The blade rotor 2 may have blade cutting surfaces or edges 22 on its top side that align with the cutting surfaces (blades) to allow smooth and continuous joint surface removal and reshaping. The blade rotor is hollow and may carry a pelvic centering pin 4 inside it. The blade rotor is able to rotate around the pelvic centering pin 4. The other end of the blade rotor 2 holds a spline linear bearing that allows the blade rotor to freely move axially while being able to carry torque at the same time. The torque may be supplied by a drill that remains outside the body during a surgical procedure.

The pelvic centering pin 4 is a long, thin bone screw that is screwed on the second side of the joint, shown at the center of the acetabulum in the pelvis in FIG. 1. The pin can be placed through a path in the femur. The pitch of the thread may be less than the thickness of the pelvis at the acetabular cavity. The path may be generated by carefully aligned drilling of the femur under fluoroscopy. During placement, the pin 4 may be guided through the femoral neck 38 and the center of femoral head 32 to the pelvis 3. The threads 40 of the head of the pelvic centering pin 4 may be self-cutting so that drilling of the pelvis 3 is not required. The pelvic centering pin may be configured of a length to extend only through the joint surface and bony portion of the pelvic bone.

A bone screw (trochanter screw) 5 holds the device in the femur 6. The trochanter screw 5 is hollow to accommodate the blade rotor 2 and the pelvic centering pin 4. The trochanter screw has self-cutting threads. The trochanter screw 5 holds an attachment port 42 configured to allow the distractor linear actuator module (containing the distractor; 7) to be attached.

The distractor linear actuator module 7 is fixed on the bone (trochanter) screw 5 which in turn is fixed on the femur 6. The distractor linear actuator module does not rotate. The rotating hollow distractor shaft 8 carries torque from the drill 13 through the flange 14 to the spline 16 that in turn delivers torque to the blade rotor 2.

The linear bearing 9 allows axial motion of one portion of the distractor. The distractor may have a 2-way actuator, such as a pneumatic actuator, to act on the bone screws and thereby distract or compress the joint space of the joint. The distractor may have a compression air chamber 11 that may supply a compressive force and a distraction air chamber 12 that may supply a distraction force, separated by a two-way piston 17, 18 as shown in FIG. 1. Input of air from the distraction air port 48 into the distraction air chamber 12 may cause distraction of the joint space. The joint may need to be distracted (FIG. 2) in order to assemble or disassemble a reaming and shaping device. Input of air from air port 48 into the compression chamber 11 may cause compression of the joint space. The distractor may distract the hip joint without interrupting the joint capsule. Ball bearings 19, 20 allow for rotation of the hollow distractor shaft 8.

The drill 13 connects to the distractor linear actuator module 7 at the drill flange 14 to deliver power from the drill into the distractor linear actuator module 7. The centering pin restrictor 15 locks the end of the pelvic centering pin 4 axially so that it can transmit an axial load to cause compression or distraction of the joint. The spline keyway 16 transmits torque but allows free axial positioning of the blade rotor 2 so that it can accommodate a bump or asphericity of the bone during the shaving process.

The cutting surfaces 1, blade rotor 2, hollow distractor shaft 8, drill 13, flange 14, and spline 16 may all rotate. The bone screw 5, distractor 7, linear bearing 9, distractor piston 17, 18, and ball bearing are fixed to the bone (femur) to allow for rotation of the distractor shaft 20. In one embodiment, the cutting surfaces 1 and blade rotor 2 are caused to rotate by torque supplied by the drill and are non-axially attached to other parts of the distractor and the drill.

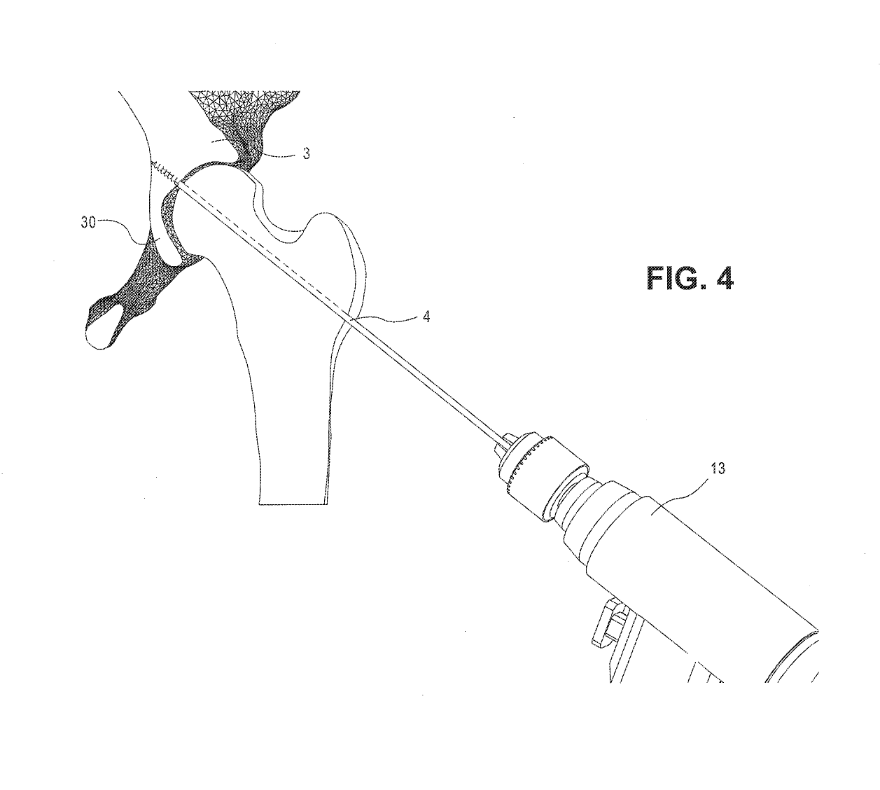

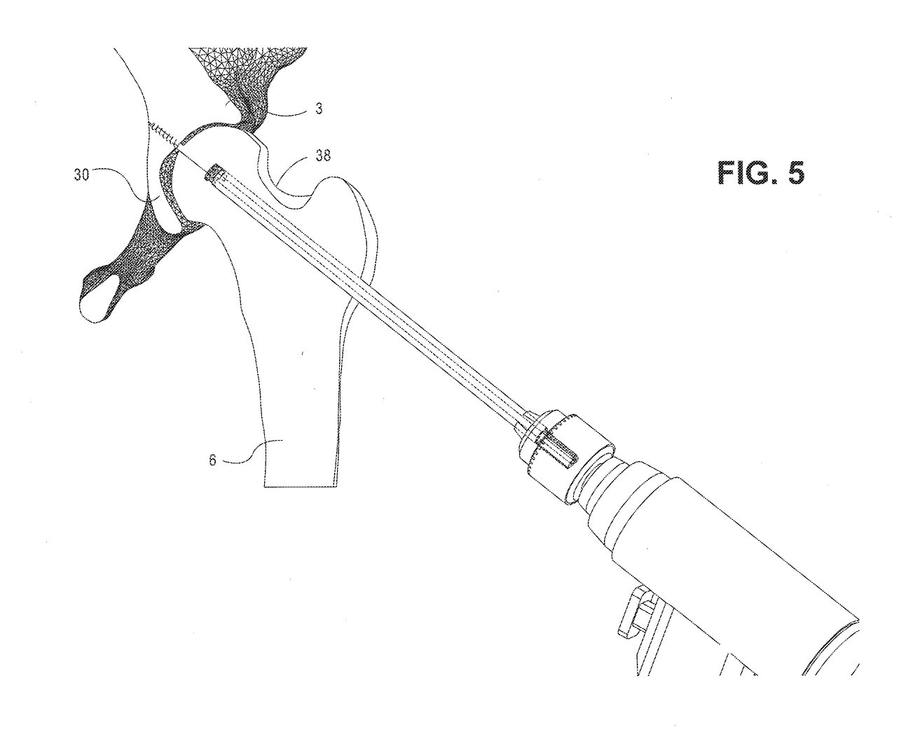

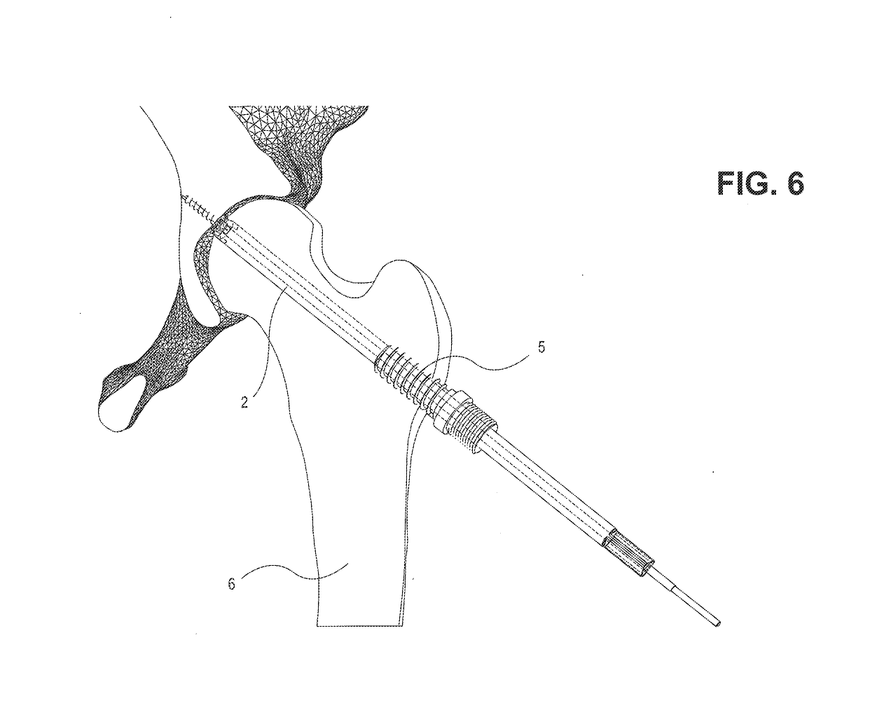

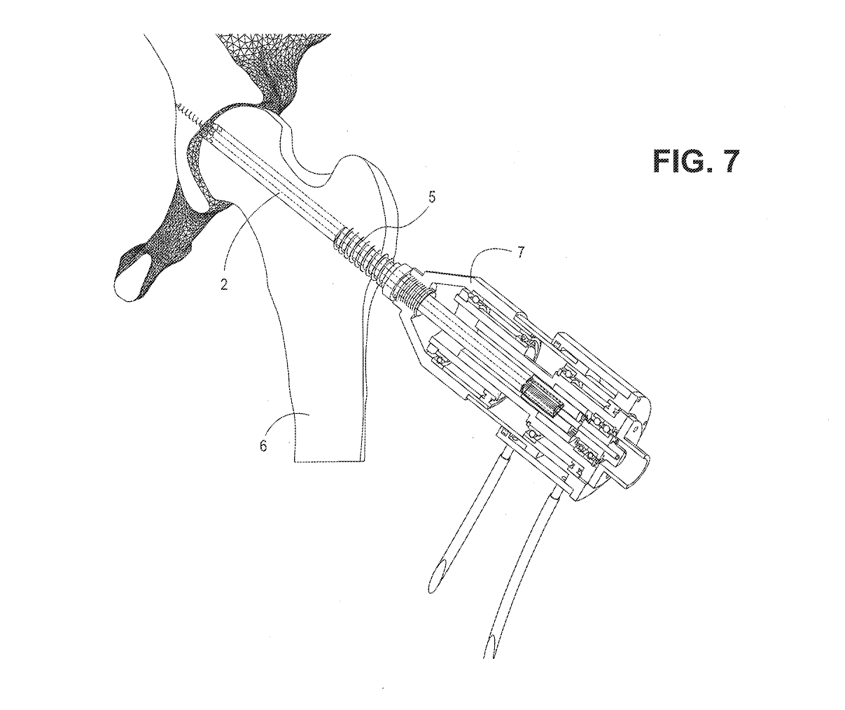

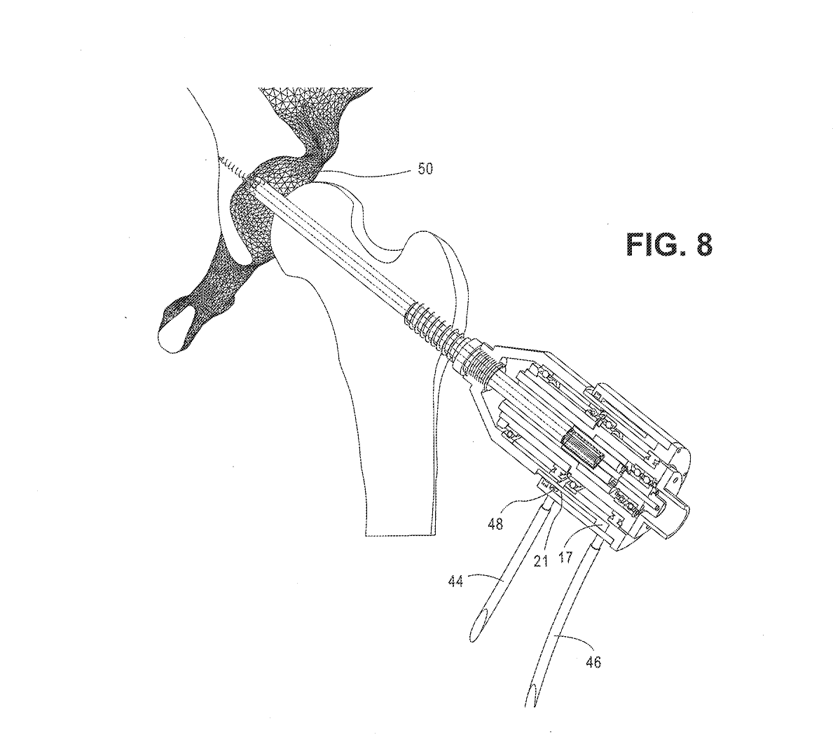

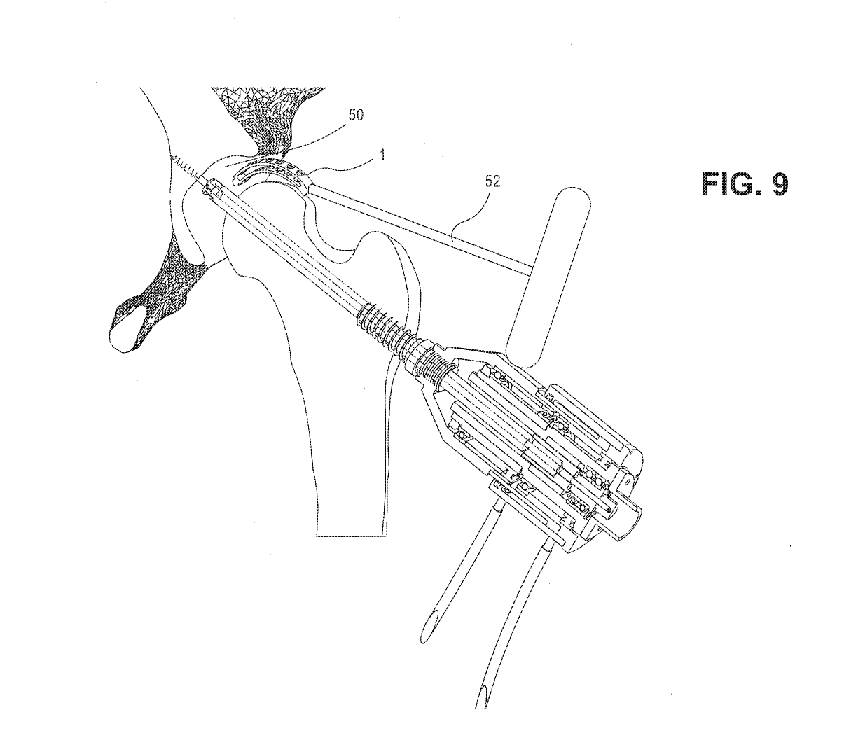

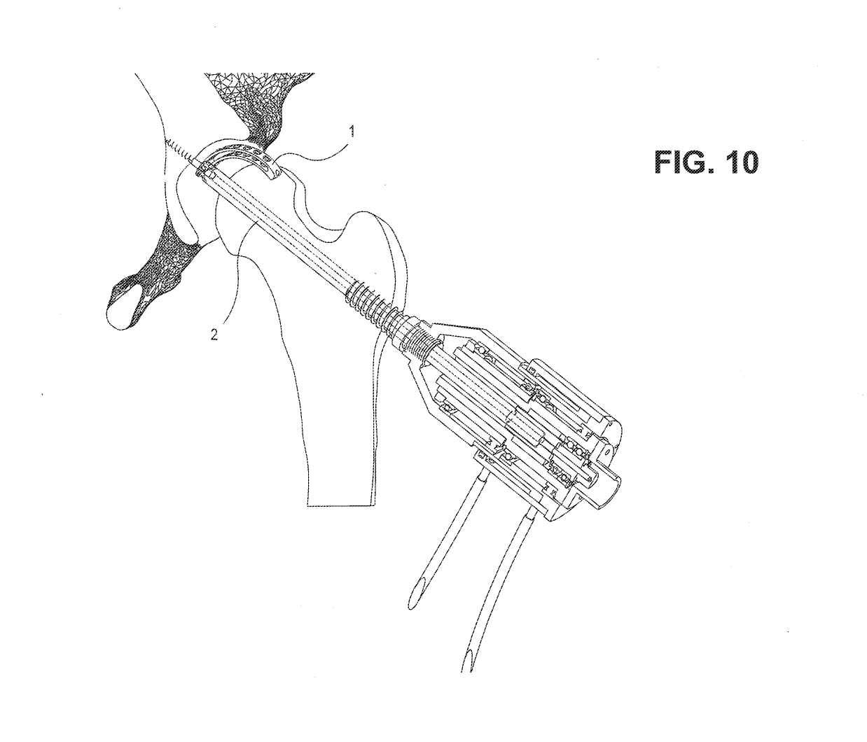

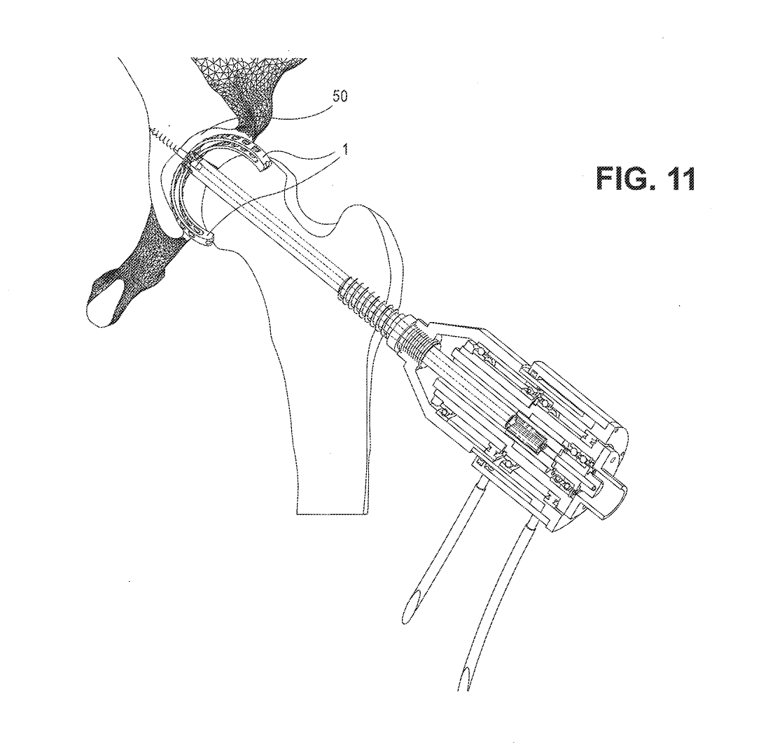

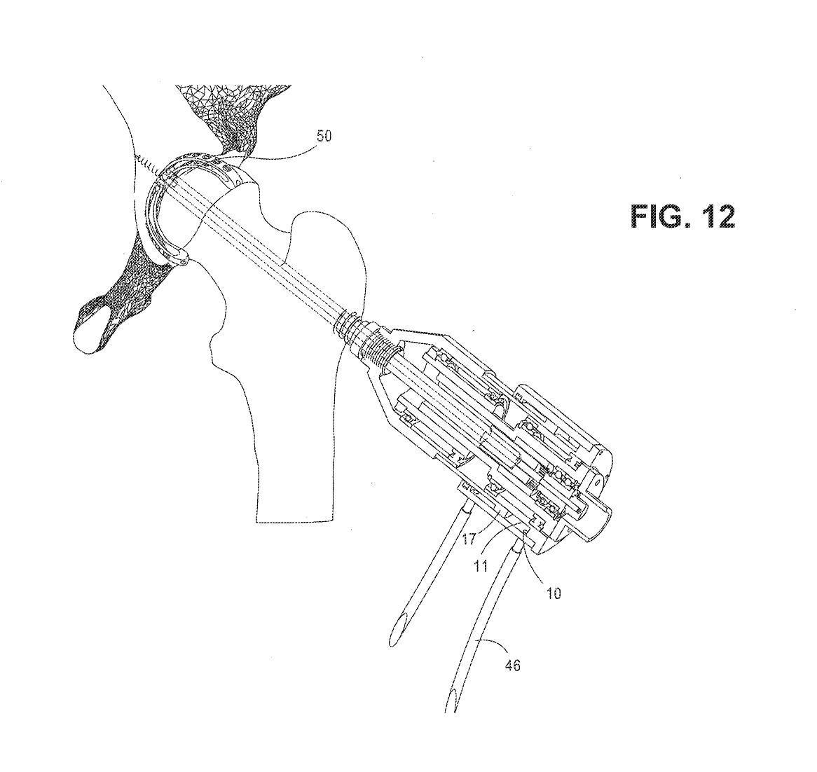

FIGS. 4-13 show assembly of a reaming and shaping device in a joint according to one embodiment of the disclosure. A small incision may be made in the skin. A camera may be placed and a field of view established. A path may be drilled through a femur 6 (e.g., under 2-plane fluoroscopy) using a drill bit (not shown) or pelvic centering pin 4 and drill 13. A pelvic centering pin 4 may be screwed into the acetabulum 30. As shown in FIG. 5, the blade rotor 2 may be inserted over the pelvic centering pin 4. As shown in FIG. 6, the drill 13 has been removed and a trochanter screw 5 is inserted into the femur 6 over the blade rotor 2. As shown in FIG. 7, a distractor linear actuator module 7 may be attached to the trochanter screw 5. As shown in FIG. 8, air from air supply 44 goes through air port 48, pressurizing the distraction chamber 12, moving piston 17 to distract joint space 50. FIG. 9 shows that a cutting surface (blade) 1 may be inserted into the joint space 50 using a blade handle 52. FIG. 10 shows the cutting surface 1 coupled to the blade rotor 2. The coupling may be by any suitable means that allows the cutting surface to rotate and move inward and outward, such as a slide-to-clip mechanism. FIG. 11 shows several cutting surfaces 1 coupled to the blade rotor 2 in the joint space 50. FIG. 12 shows compression of the joint surface 50 after addition of air from air supply 46 through air port 10, pressurizing compression chamber 11, and joint space 50.

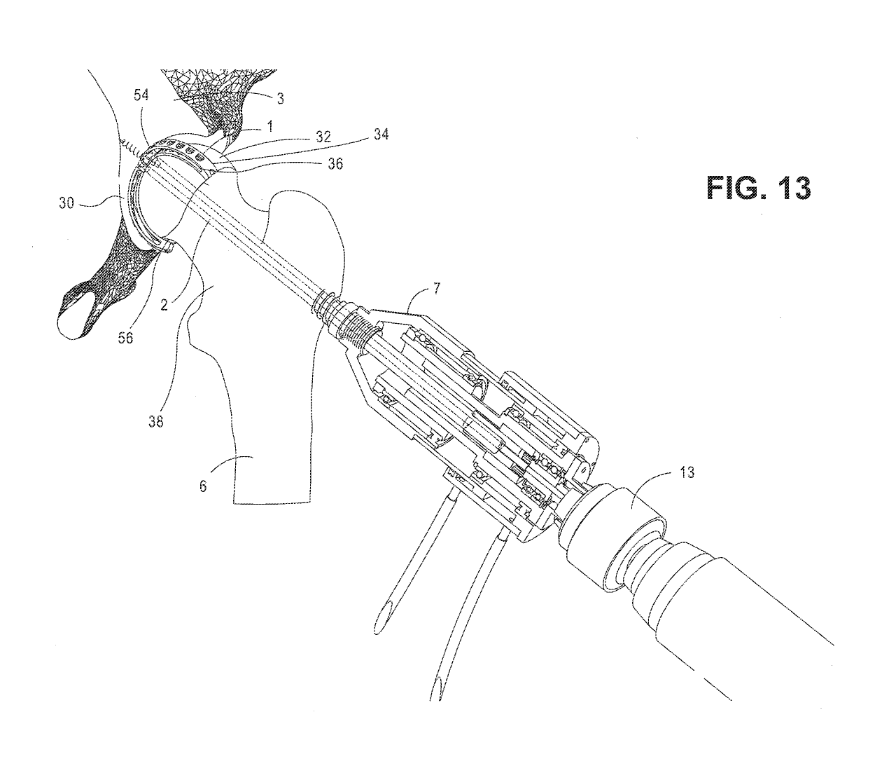

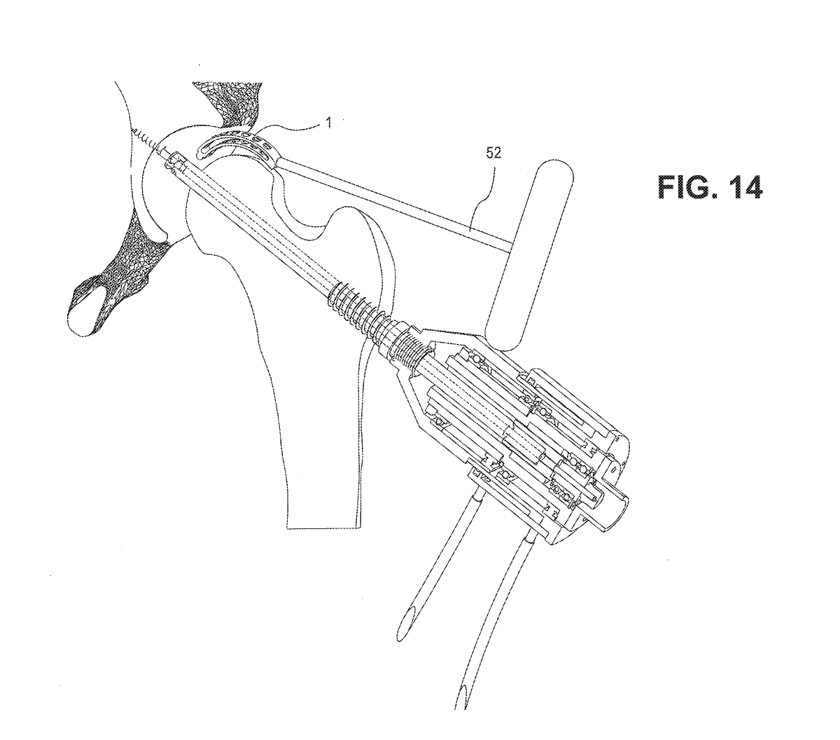

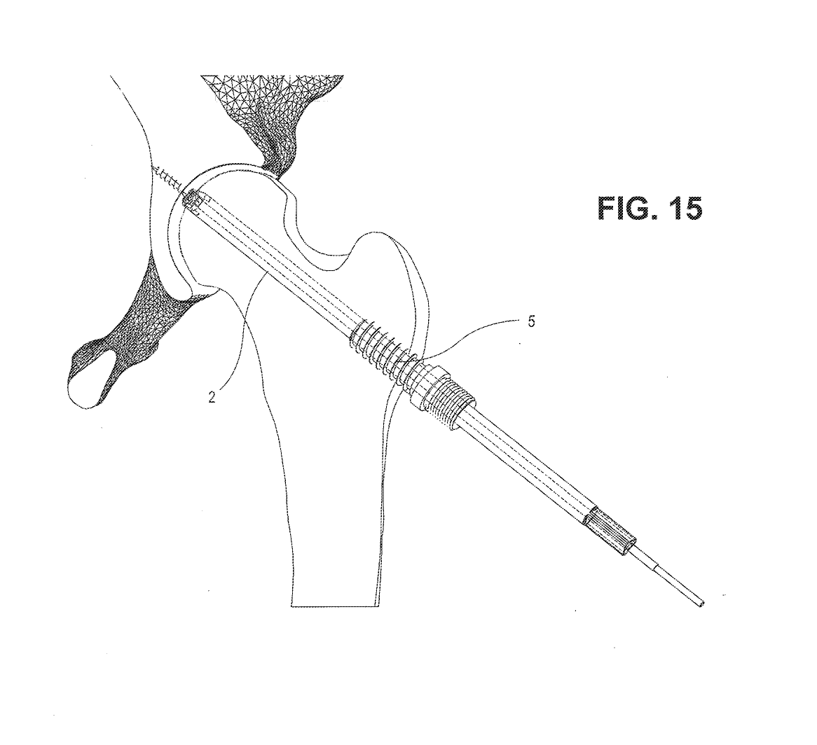

FIG. 13 shows attachment of drill 13 to the distractor linear actuator module 7 and delivery of torque through the linear actuator module 7 to rotate the blade rotor 2, which in turn rotates the cutting surfaces 1. The rotation of the cutting surfaces 1 causes the first facial surface 34 of the cutting surface to ream the acetabulum of the pelvis 3 at the same time that the second facial surface 36 shaves and shapes the femoral head 32. Pressure from the acetabulum onto the first facial surfaces causes the cutting surfaces to pivot around an attachment end 54, and the free end 56 of the cutting surfaces 1 to move towards the central blade rotor axis and to grip the surface of the femoral head. Continued pressure from the acetabulum as reaming, shaving and shaping continues causes a decrease in the angle formed between the cutting blade 1 and the axis of the blade rotor 2. In some embodiments, the femoral head may be shaped into a sphere by the shaving and shaping steps. In another embodiment, the joint may be shaped into an ellipsoid, cylindrical, chamfered cylindrical, or tapered cylindrical shape. Air acts as a pressurizer and as a damper/spring to accommodate the shape irregularities of the bone and smooth the shaving process. As shown in FIG. 14, reaming is complete. Blade handle 52 may be arthroscopically inserted through a small (e.g., 30 mm) hole to remove a cutting surface blade 1, and this step may be repeated until all of the cutting surface blades are removed. As shown in FIG. 15, the distractor linear actuator module and the remainder of the reamer/shaper parts have been removed, leaving the trochanter screw 5 and blade rotor 2 to be removed.

In another embodiment, a shaper device to shape only a single side of a joint surface utilizes cutting surfaces that move relative to the joint surface. In another embodiment, the shaper re-shapes greater than 30° of a joint surface. In another embodiment, the shaper reshapes more than 180° (e.g., 181°-250°) of a joint surface. The joint surface may be roughly spherical or otherwise protruding. The shaper device may have a guide pin to center the shaving process and a cannulated drill that operates over the guide pin.

The cutting surfaces may be separated from one another during insertion of the device in the joint to allow the device to fit over the joint end. The cutting surfaces may be brought closer together around a spherical or protruding joint surface in preparation for or during use (clam shell design). The cutting surfaces may close over the joint surface and grip the surface during use.

For brevity, the disclosure shows the device configured for use in a hip joint, although the device can be used on any suitable movable joint (e,g., ball-and-socket, condyloid or hinge structure such as finger, hip, knee, shoulder, toe, etc). The inner shape of the shaper may be any suitable shape corresponding to the joint surface to be shaped (e.g., spherical, elliptical tapered cylinder, chamfered cylinder). In one embodiment, a femoral shaper may be configured to shape a sphere.

The shaper may re-shape the joint surface to a spherical geometry by removing the cartilage and a small amount of the subchondral bone. In one embodiment, the shaper device may have one cutting surface. The shaper device may have a plurality of cutting surfaces. In one embodiment the shaper has three cutting surfaces.

The cutting surfaces may be arc shaped. The clam shell cutting surface of a device may comprise one or more separate shell cutting surfaces. The cutting surfaces may be any longitudinal length able to change a shape of the joint. The cutting surfaces may extend so as to encompass the joint surface (e.g. from 15-125°). In one embodiment, the cutting surfaces are longitudinal spherical arcs extending to 91-125° as measured from the top, central axis (North Pole) and extend over the sides of the joint surface. As cutting occurs, the clam shells continuously press down on the femoral head, causing additional shaving of the femoral head surface. The cutting surfaces may have a collar region configured to protect a portion of the bone surface from unwanted shaving at the bottom edge of the cutting surface. Undesirable shaving may lead to bone fractures and/or damaged blood vessels. In one embodiment, the collar region protects the femoral neck.

One or more (e.g., 2-6) clam shell cutting surface(s) may make up the clam shell shaper. These shells may cover a total of 1-100% of the circumferential (lateral) area of a sphere. The cutting portion or edge of the cutting surface may be any shape or material effective for shaving or trimming joint surface material. The cutting portion may be an abrasive surface (e.g., a file-like shaving surface o multiple large or small cutting teeth (e.g., grater style).

The clam shell cutting surfaces may be closed over the femoral head via a clamping mechanism. The clamping mechanism may cause the cutting surfaces to grip the joint surface. A stop mechanism may prevent over-reaming. The stop mechanism may be a spring, a nut, or a mating of the clam shell cutting surfaces. The clam shell cutting surfaces may be engaged and disengaged (i.e., closed and opened) from the femoral head surface via a locking mechanism.

The clamping mechanism for the clam shell cutting surfaces may apply continuous force to the clam shells to facilitate cutting of the femoral head surface. As shaving occurs, the clamping mechanism may cause the clam shells to close in against the femoral head and expose more of the femoral head surface to the clam shell cutting surfaces. The clamping force may be controlled through any suitable mechanism to cause the cutting surfaces to grip the surface in order to shave the surface and to release the surface to allow the device to be removed. In one embodiment a hydraulic system may control the clamping. In another embodiment, a pneumatic system may be used to control the clamping. In another embodiment a spring may be used to control the clamping. The spring force may be constant during shaving or may decrease during shaving. In one embodiment, the spring may be controlled by the surgeon. One embodiment of a surgeon-controlled spring force may utilize a handle to adjust the spring length using hand pressure. Another embodiment of a surgeon-controlled spring force may be a pressure-activated (squeezing force) sheath around nut that regulates the spring length.

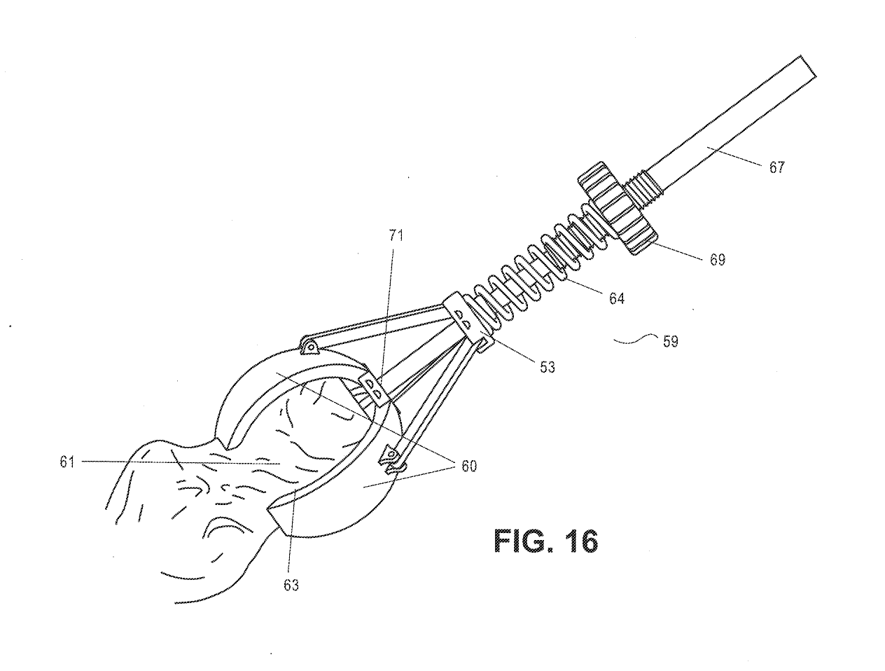

FIG. 16 shows a femoral shaper 59 on a femoral head 61. The shaper 59 has spherical or arc shaped cutting surfaces (clam shells; 60). As shown, the cutting surfaces 60 are longitudinal spherical arcs extending 91-125° from the top (central axis or North Pole) of the device head 71. The cutting surfaces 60 have one or multiple cutting edges or portions 63 on its inner surface. Cutting surfaces 60 couple with a blade rotor 67 which is configured to couple with a drill. The cutting surfaces 60 are forced by the spring 64 through the leg acceptor/stop mechanism 65 onto the femoral head 61. The tension of the spring 64 is controlled by a spring adjuster 69 which sits on the blade rotor 67. The spring adjuster 69 may be manually adjusted by the surgeon during the shaving process. As the blade rotor 67 turns the cutting surfaces 60, the cutting surfaces 60 trim the femoral head 61, and the tension from spring 64 forces the cutting surfaces 60 to continuously grip the femoral head. The leg acceptor/limiting nut 53 stops the device from shaving too much.

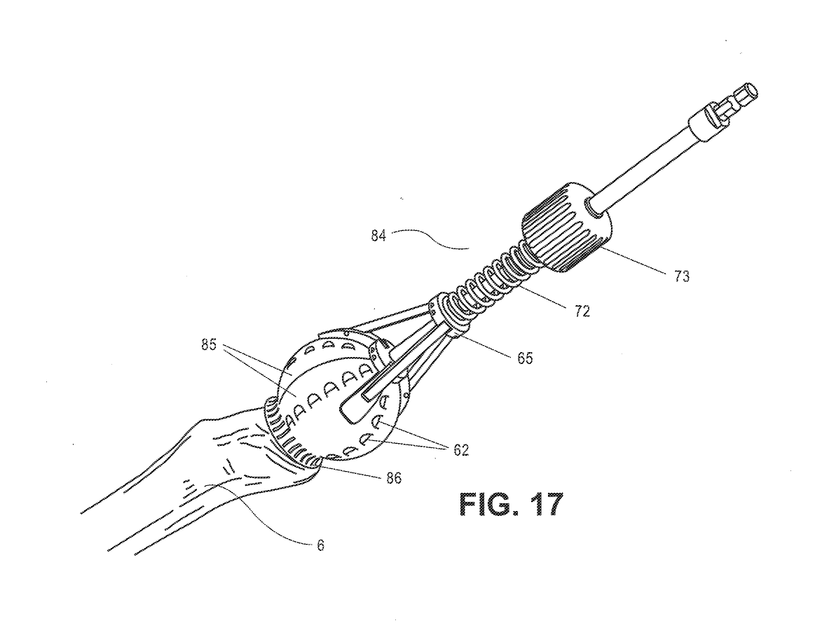

FIG. 17 shows another embodiment of a femoral shaper 84 on a femur 6. The shaper has a plurality of arc shaped cutting surfaces 85, each with a collar region 86 and a plurality of cutting edges or portions 62. The cutting surfaces cover about 30-40% of the circumferential (lateral) area of the femoral head. The spring 72 controls the tension placed on the cutting surfaces 85. The nut 73 is used to control the force transmitted to the cutting surfaces 60. The cutting surfaces 85 are prevented from overcutting because the cutting surfaces 85 are configured to abut each another when the cutting surfaces 85 have reached the desired position for the optimal shape of the joint surface and the leg acceptor/stop mechanism 65 limits the inward movement of the cutting surfaces 85.

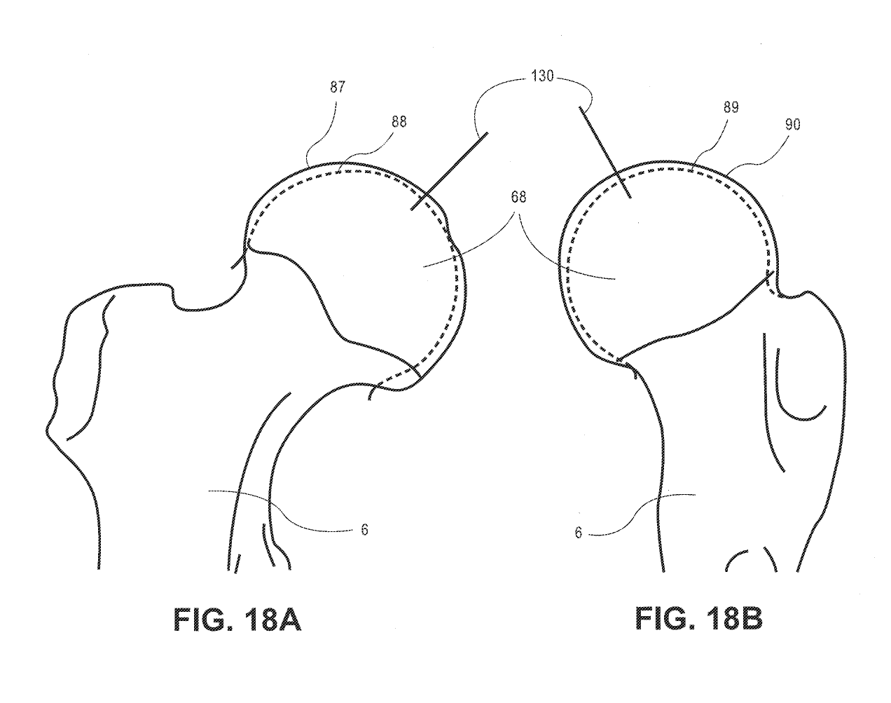

FIGS. 18A and B show preparation of a joint surface for treatment with a device from the disclosure. A guide pin 130 is centered on a femoral head 68 of a femur 6 for positioning a shaping device on the femur. Two joint surfaces are shown with the starting shape of the joint 87, 89 and the ending shape of the femoral shaper 88, 90 that will be used to shave and shape the joint surface. The joint material to be removed has surface irregularities and its thickness varies from about 0.5 mm to 2 mm.

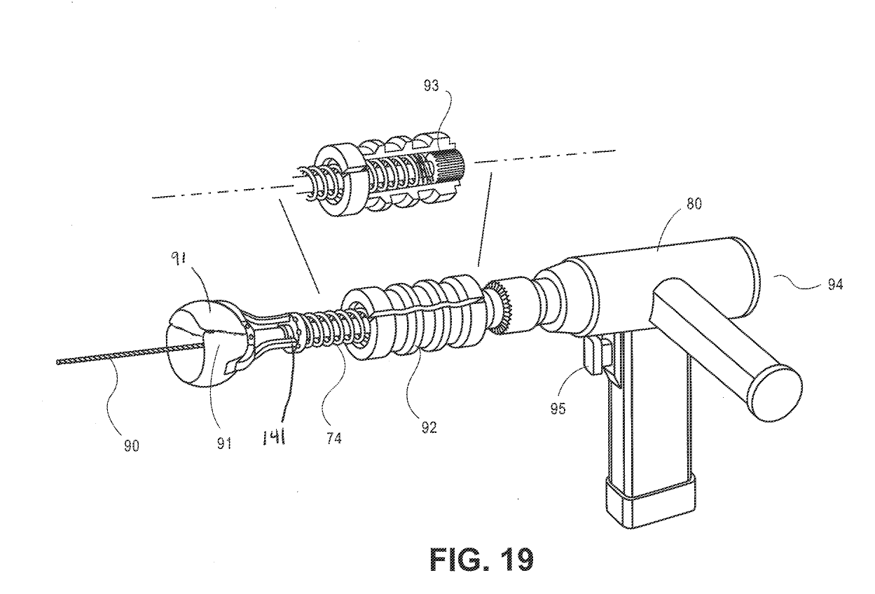

FIG. 19 shows a femoral shaper 94 with a spring mechanism 74 and pressure sheath 92 over a nut 93 to control the force placed on the cutting surfaces 91. The force is configured to keep the cutting surfaces 91 in contact with and gripping a bone (not shown in this view) during use. The pressure sheath 92 is configured to be manually adjusted during device use to clutch and control the underlying nut 93. By increasing the pressure on the pressure sheath 92, the friction between the sheath and the nut increases, causing the nut to get screwed further and to regulate the length of spring mechanism 74 to prevent pushing cutting surfaces 91 too close together. A protrusion 141 prevents over-reaming by preventing the cutting surfaces 91 from closing too far. The pressure sheath 92 is configured to provide the user with tactile feedback as to the degree of friction. The femoral shaper 94 is centered in the bone has a guide pin 140 to center and stabilize the device with the bone. The speed of drill 80 is controlled with a button 95.

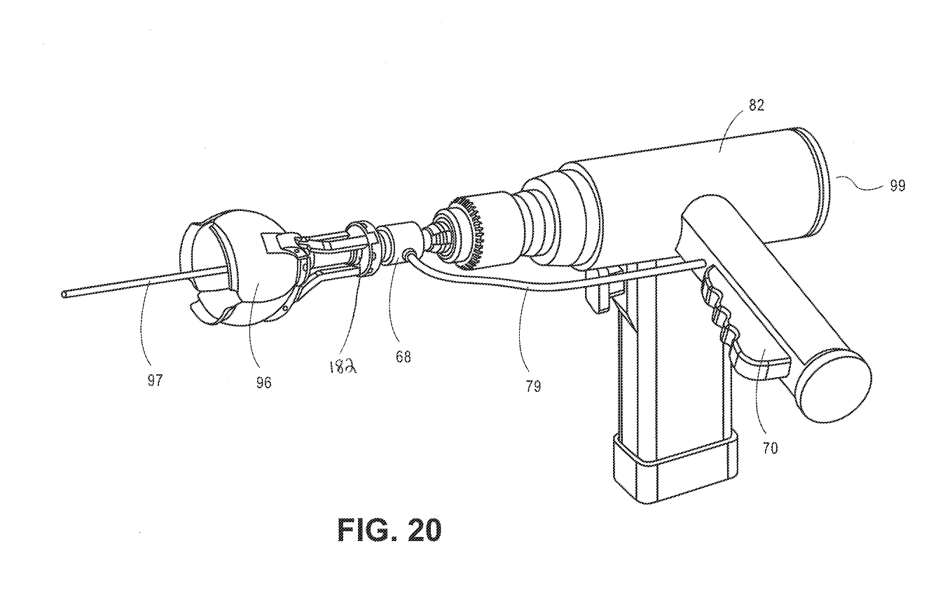

FIG. 20 shows a femoral shaper 99 with a non-rotating hydraulic piston control mechanism 68 that controls the degree of clamping or gripping of the cutting surfaces 96 with underlying bone (not shown in this view). The hydraulic piston control mechanism 68 is controlled by hand using hydraulic control lever 70 through line 79. The femoral shaper 99 has a guide pin 97 to center the device in the bone and provide stability. The device has a feature 182 to stop the cutting surfaces from over-reaming.

In one embodiment, the disclosure is a method for shaving a joint surface, comprising:

1. Placing a guide pin in the femoral head at the point of the central axis of shaving (e.g., North Pole of the femoral head)

2. Placing a cannulated drill on the guide pin with the clam shell cutting surfaces fully open.

3. Placing shaper against the femoral head, and activating the clam shell closing mechanism

4. Activating the shaver with the drill mechanism

5. Applying/irrigating the shaver and/or joint surface with a treatment solution (e.g., cool saline) during the shaving.

6. As cartilage and bone are removed by the cutting teeth, closing the clam shell gradually around the femoral head, thereby shaping the femoral head into a spherical shape.

7. Shaving stops once the clam shell has fully closed.

8. Opening the clam with the clam shell closing mechanism; removing the shaver and guide pin.

The shaver may be activated using hydraulic, pneumatic or mechanical means.

In another embodiment of a joint surface shaper, the device is hand-held, manually operated, and has a low profile. The device may be introduced into the joint space without fully rotating the bone outwards which reduces tissue damage. For brevity, the disclosure shows the device configured for use in a hip joint, but it can be used on any suitable movable joint (e.g., ball-and-socket, condyloid or hinge structure such as finger, hip, knee, shoulder, and toe). The device may be placed on the hip joint surface through a small incision in the skin.

In one aspect of the disclosure, the device lacks a pelvic centering pin and may be moved or rotated to shave different regions of the joint surface. The shaper may have any final shape (e.g., sphere, ellipse tapered cylinder, chamfered cylinder) corresponding to a desired shape of the joint. In one embodiment, the shaper is configured to generate a spherical shape.

The shaper may have at least one cutting surfaces. In some embodiments, the shaper has 2, 3, or more cutting surfaces.

The cutting surfaces may be any shape to shape the joint surface. In one embodiment the cutting surfaces are in the shape of a wide arc and the edges of the cutting surfaces are configured to mate together. The cutting surfaces may be coupled to the joint surface shaper so that the cutting surfaces pivot around an attached end during device use. In one embodiment, the cutting surfaces resemble clamshells and close together.

The cutting surfaces may have cutting portions (or edges). The cutting portions may take any form able to remove cartilage, bone, or other joint materials from the surface of the joint. The cutting portions may be shaped and sized to grate or shave joint materials. The cutting portions may be a plurality of teeth which may have directionality, or may be similar to a shark's teeth and may have no directionality. The cutting surfaces may be caused to vibrate using a vibrator or piezoelectric element to increase the efficiency of joint surface material removal.

The size of the head of the shaper may be changed during use. The shaper head may start in an expanded position and be made smaller during device use to allow insertion into the joint and gradual trimming of the joint surface to generate a final, desired shape. The size of the shaper head may be controlled by a restraint system that encircles or otherwise connects a cutting surface with a controller, and causes the cutting surfaces to expand outwardly or move inwardly. In one embodiment, the restraint is a wire-rope encircling a plurality of cutting surfaces and a trigger-handle mechanism controls the degree of restraint.

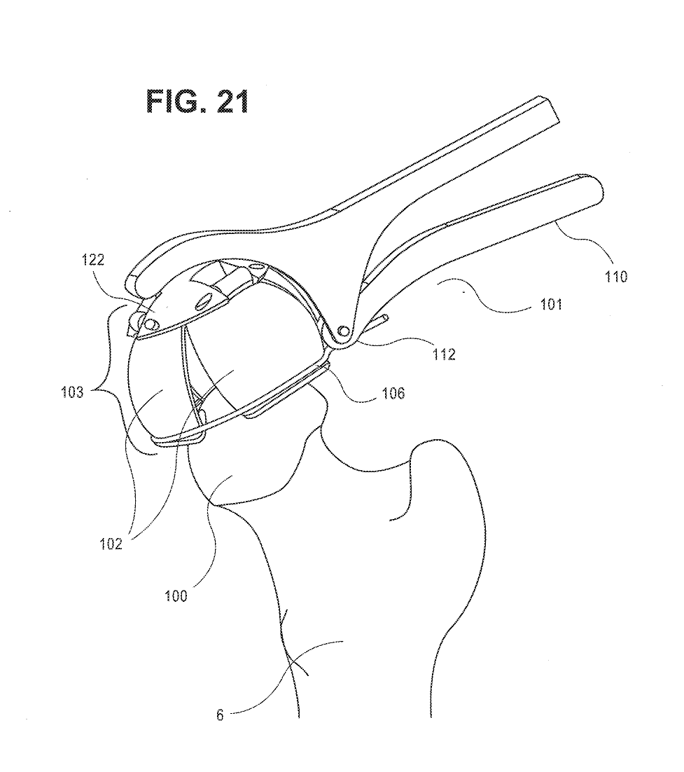

The femoral shaper may be moved over the joint surface to shave and shape a joint surface larger than the area covered by the device. The shaper may be moved in any direction (up, down, left, right) where shaping or shaving is desired. In one embodiment, the shaper may be moved as far as 30-45° from a starting axis or axis of a joint. FIGS. 21-25 show embodiments of the hand-held shaper.

FIG. 21 shows a femoral shaper 101 being placed on the femoral head 100 of a femur 6. Cutting surfaces (blades, 102) are coupled to a headpiece 122 which is connected to the trigger-handle 110. The trigger-handle 110 is shown in a more open (expanded) position so that the controller 112 allows a longer length of wire-rope 106 to encircle cutting surfaces 102 so that the cutting surfaces (or blades 102) are separated to allow insertion of the device head 103 over the femoral head 100. When the trigger-handle is squeezed, the wire-rope is pulled and the length of the wire-rope encircling the cutting surfaces is shortened, moving the cutting surfaces 102 toward/onto the femoral head and shaving the joint surface.

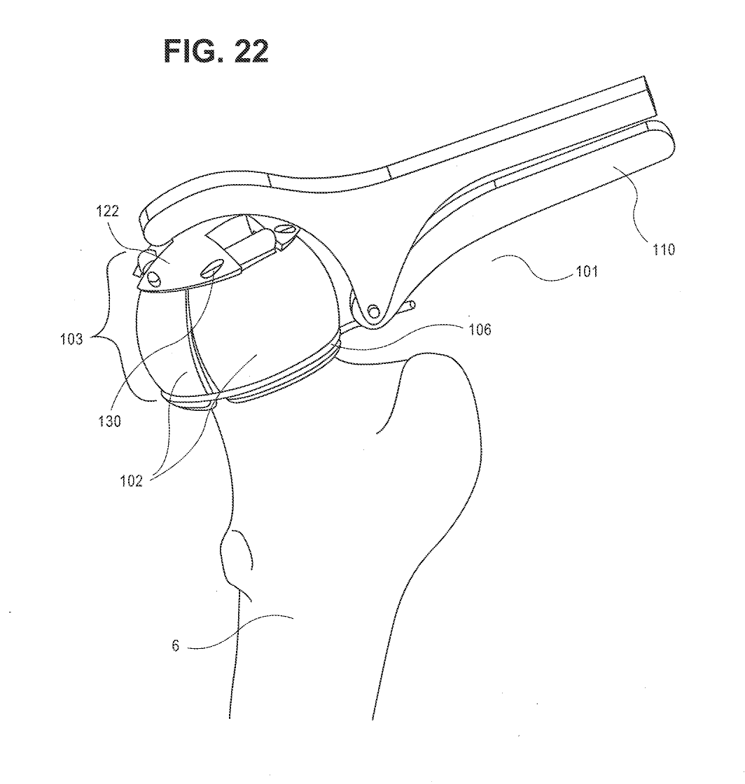

FIG. 22 shows a femoral shaper 101 like the one in FIG. 21 closed over the head of femur 6. Squeezing the trigger-handle 110, which is shown in a closed configuration at the end of the squeezing motion, moved the cutting surfaces 102 inwardly while shaving and shaping the surface of the joint under the device. The cutting surfaces 102 have pivoted around the blade attachment pin 130 to close over the femoral head and create the desired surface shape and size under the head 103 of the shaper 101. Further inward motion of the cutting surfaces is prevented to preclude over-reaming (removal of more material than desired) of the surface. In one embodiment, the cutting surfaces meet or mate and prevent further inward motion of the cutting surfaces. In another embodiment a portion of the cutting surface may abut the headpiece 122 which thereby prevents further inward motion of cutting surface. The wire-rope 106 encircling the cutting surfaces is in a shortened position.

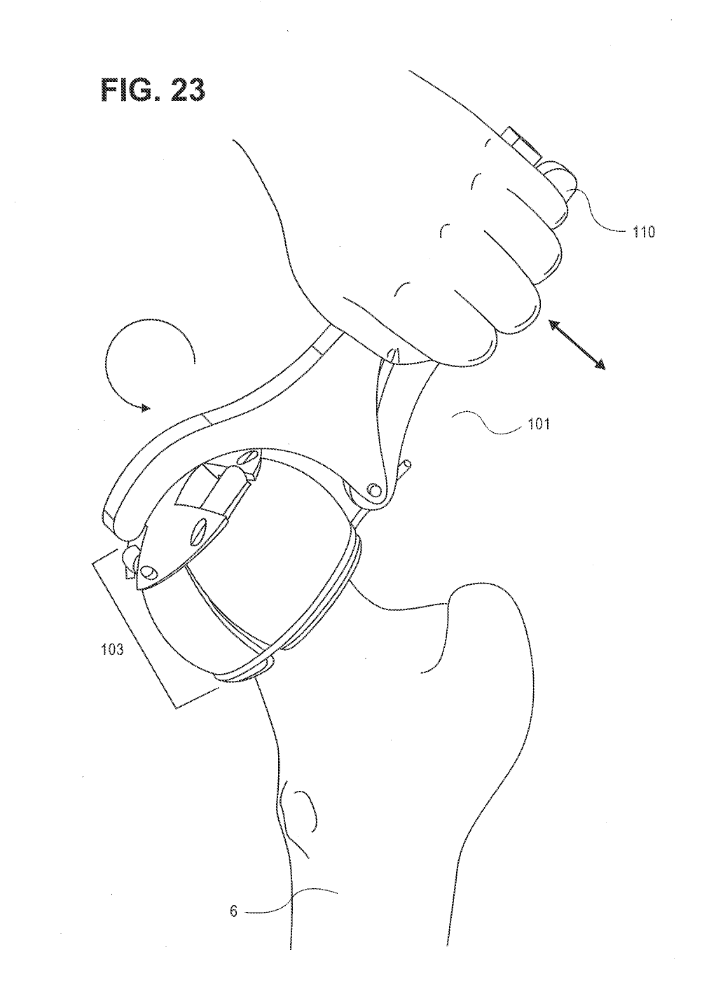

FIG. 23 shows the femoral shaper 101 shown in FIG. 22 moved to a different position on the head of the femur 6. The head 103 of the shaper was first expanded to a more open position. Next the shaper 101 may have been moved while the head 103 was being closed and the surface simultaneously shaved or the shaper 101 may have first been moved to a new position and then the trigger-handle 110 squeezed to close the head 103 and shave a different but overlapping portion of joint surface.

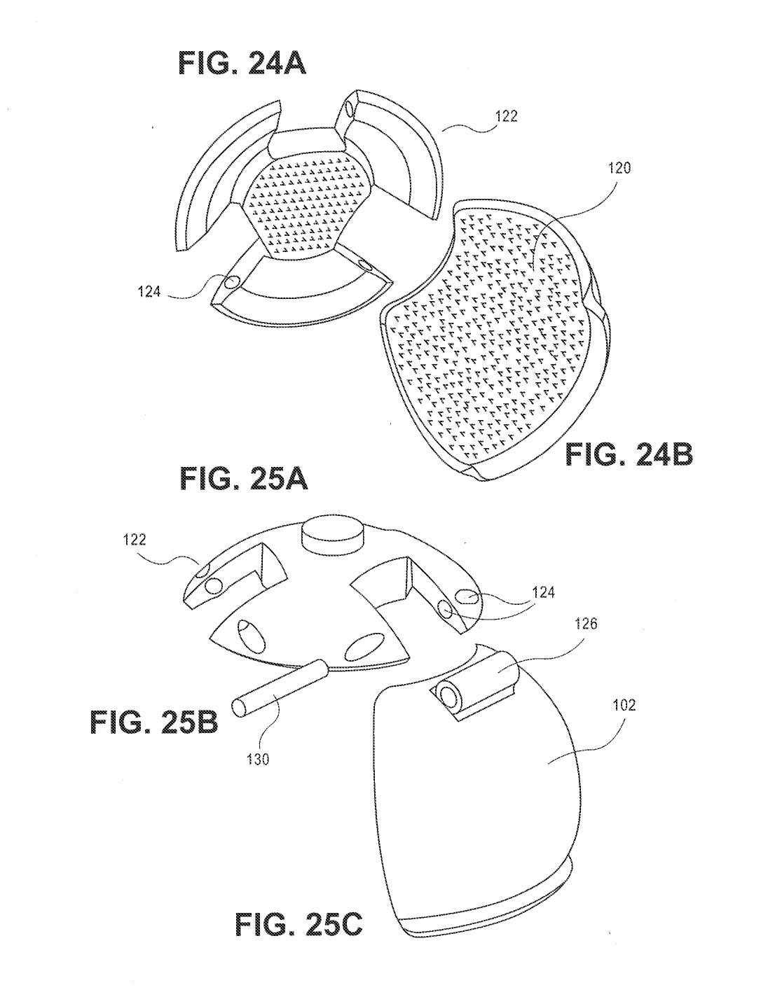

FIG. 24A-B show a bottom/inside view of the headpiece 122 and an inside view of cutting surface 120 of the head of a femoral shaper 101. The headpiece 122 has slots 124 for accepting the blade attachment pin 130. The central portion of headpiece 122 and the inner surface of cutting surface 120 have roughness or teeth for shaving or grating and shaping the surface of the joint.

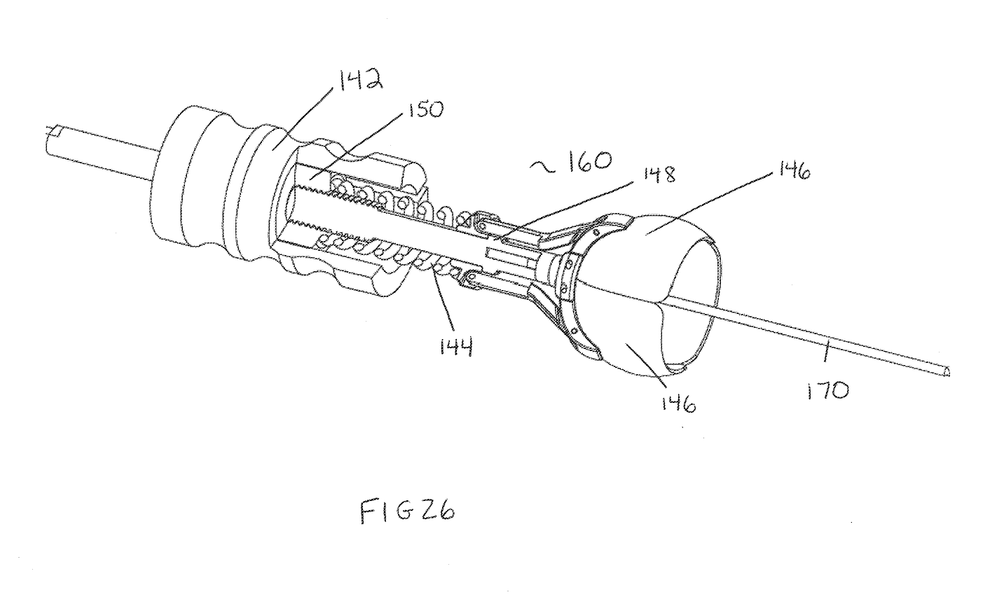

FIG. 25A-C show an outside view of the headpiece 122, cutting surface 102, and blade attachment pin 130. The headpiece 122 has slots 124 for accepting the blade attachment pin 130. The cutting surface 102 has a pin acceptor 126 through which the blade attachment pin 130 couples the cutting surface 102 to the headpiece 122.

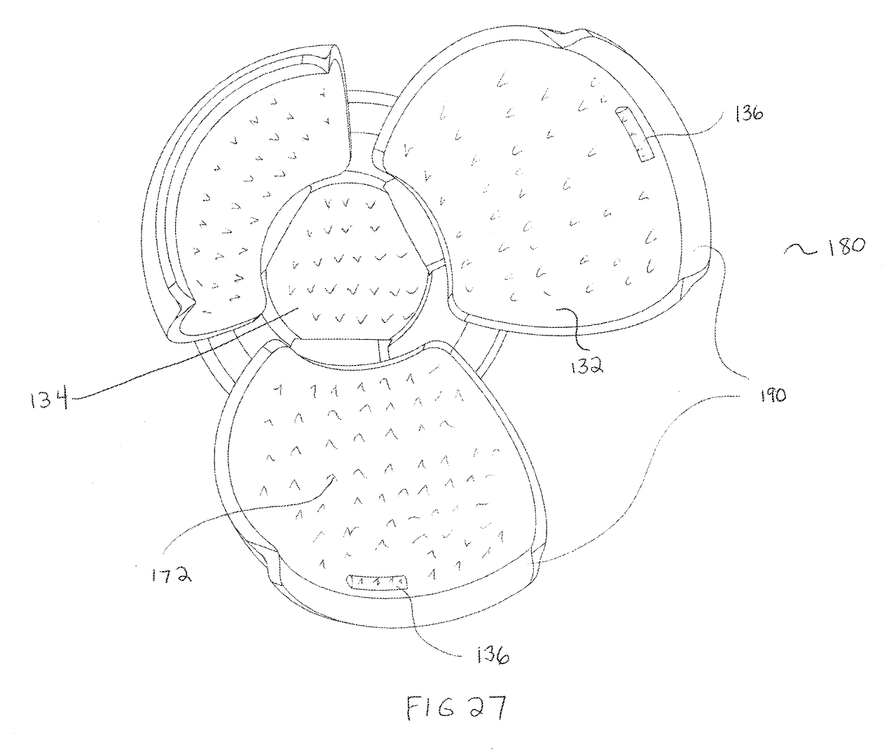

FIG. 26 shows a detail view of a femoral shaper 160 similar to the device in FIG. 19 with a spring mechanism 144 and pressure sheath 142 over a nut 150 to control force placed on the cutting surfaces 146. The force is configured to keep the cutting surfaces 146 in contact and gripping a bone (not shown in this view) during use. The pressure sheath 142 is configured to be manually adjusted during device use to clutch and control the underlying nut 150. By increasing the pressure on pressure sheath 142, the friction between the sheath and the nut increases, causing the nut to get screwed further and regulate the spring length before pushing cutting surfaces 146 too close. A feature 148 (e.g. protrusion or indentation) prevents over-reaming by preventing the cutting surfaces 146 from closing too far. The pressure sheath 142 is configured to provide the user with tactile feedback as to the degree of friction. The femoral shaper 160 is centered in the bone has a guide pin 170 to center and stabilize the device with the bone.

FIG. 27 shows an inside/bottom view of a femoral shaper 180 similar to the shaper shown in FIG. 21. The inside of cutting surface 132 and headpiece 134 have cutting teeth 172 configured to shave and shape a joint surface. The inside of cutting surface 132 has a protrusion 136 configured to create a groove or depression in a joint surface. The cutting surfaces have a collar-forming region 190 to prevent over-reaming at the femoral neck.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A method of modifying a shape of a joint surface of a mammalian joint comprising:
placing a cutting tool at the joint surface, the cutting tool having a cutting surface extending over more than 90° of the joint surface;
moving the cutting surface with respect to the joint surface; and
removing joint tissue simultaneously from more than 180° of the joint surface to thereby modify the shape of the joint surface.

2. The method of claim 1 wherein the cutting tool comprises a plurality of cutting surfaces and moving comprises moving the plurality of cutting surface with respect to the joint.

3. The method of claim 1 further comprising assembling at least a portion of the cutting tool in situ in a joint space of the joint.

4. The method of claim 1 wherein the joint surface is a first joint surface corresponding to a first bone, the method further comprising simultaneously removing joint surface tissue from a second joint surface corresponding to a second bone with the cutting tool.

5. The method of claim 1 further comprising forming a groove or depression in the joint surface.

6. The method of claim 1 wherein the cutting tool defines a central axis and moving the cutting surface comprises rotating the cutting surface about the central axis.

7. The method of claim 1 wherein the cutting tool defines a central axis, the method further comprising moving at least a portion of the at least one cutting surface toward a point on the central axis.

8. The method of claim 1 wherein the cutting surface has an attached end defining a pivot, the method further comprising pivoting the cutting surface at the pivot.

9. The method of claim 1 wherein the cutting tool comprises a plurality of cutting surfaces and moving comprises moving the plurality of cutting surfaces.

10. The method of claim 1 further comprising, before the moving steps:
placing a guide pin in the joint;
placing a cannulated drill on the guide pin;
placing the cutting tool in the joint coaxially with the guide pin; and
activating the cutting tool.

11. The method of claim 1 wherein moving the cutting surface comprises gripping the joint surface with the cutting surface.

12. The method of claim 1 wherein moving the cutting surface comprises releasing the cutting surface from the joint surface.

13. The method of claim 1 further comprising distracting the joint before the placing step.

14. The method of claim 1 further comprising compressing the joint after the placing step.

15. The method of claim 1 further comprising applying a treatment solution to the cutting tool after the placing step and during the activation step.

16. The method of claim 15 wherein the applying step further comprises applying cooled saline.

17. The method of claim 1 further comprising engaging a stop mechanism to prevent over-reaming of the joint surface.

18. The method of claim 1 wherein the joint is selected from the group consisting of finger joints, hip joints, knee joints, shoulder joints, and toe joints.
